SEQUENCE LISTING (25 sequences)

SEQ ID NO: 1 - DNA, 20 bp, Artificial Sequence
  synthetic antisense oligomer
ctgcacctccatccttgagc

SEQ ID NO: 2 - DNA, 20 bp, Artificial Sequence
  synthetic antisense oligomer
ctgcacttccatccttgagc

SEQ ID NO: 3 - DNA, 18 bp, Artificial Sequence
  synthetic antisense oligomer
gcacttccatccttgagc

SEQ ID NO: 4 - DNA, 17 bp, Artificial Sequence
  synthetic antisense oligomer
gcacttccatccttgag

SEQ ID NO: 5 - DNA, 23 bp, Artificial Sequence
  synthetic antisense oligomer
ctgcacttccatccttgagcttc

SEQ ID NO: 6 - DNA, 21 bp, Artificial Sequence
  synthetic antisense oligomer
gtctgtagcttccaccgaatt

SEQ ID NO: 7 - DNA, 21 bp, Artificial Sequence
  synthetic antisense oligomer
ggctgaatcttccacctactt

SEQ ID NO: 8 - DNA, 20 bp, Artificial Sequence
  synthetic antisense oligomer
ccttcccagccctaactgac

SEQ ID NO: 9 - DNA, 21 bp, Artificial Sequence
  synthetic antisense oligomer
cttaccgcatgtccccgtaag

SEQ ID NO: 10 - DNA, 19 bp, Artificial Sequence
  synthetic antisense oligomer
ctccaaactggaaagacac

SEQ ID NO: 11 - DNA, 20 bp, Artificial Sequence
  synthetic antisense oligomer
gaccctttaccttcagcggc

SEQ ID NO: 12 - DNA, 20 bp, Artificial Sequence
  synthetic antisense oligomer
ggtacttctgtttccctggg

SEQ ID NO: 13 - DNA, 20 bp, Artificial Sequence
  synthetic antisense oligomer
gtatcttacctcccagagtc

SEQ ID NO: 14 - DNA, 19 bp, Artificial Sequence
  synthetic antisense oligomer
cagcttccgggctattggg

SEQ ID NO: 15 - DNA, 23 bp, Artificial Sequence
  synthetic antisense oligomer
ccttttccttaccaggcaaggcc

SEQ ID NO: 16 - DNA, 21 bp, Artificial Sequence
  synthetic antisense oligomer
ggaagcctggagaagaagagg

SEQ ID NO: 17 - DNA, 20 bp, Artificial Sequence
  synthetic antisense oligomer
gcacttactcattgaaaacc

SEQ ID NO: 18 - DNA, 20 bp, Artificial Sequence
  synthetic antisense oligomer
gcatgcggtacctgggaagg

SEQ ID NO: 19 - DNA, 20 bp, Artificial Sequence
  synthetic antisense oligomer
ggcacttactaatgctgaag

SEQ ID NO: 20 - DNA, 19 bp, Artificial Sequence
  synthetic antisense oligomer
ccactggaactgatgtggg

SEQ ID NO: 21 - DNA, 20 bp, Artificial Sequence
  synthetic antisense oligomer
cgtttgcttacaggctgcac

SEQ ID NO: 22 - DNA, 19 bp, Artificial Sequence
  synthetic antisense oligomer
ctcgcaatctgtagggaag

SEQ ID NO: 23 - DNA, 15 bp, Artificial Sequence
  synthetic internal standard
gaggggcatcgtcgc

SEQ ID NO: 24 - DNA, 20 bp, Artificial Sequence
  synthetic internal standard
gcgacgatgcccctcaacgt

SEQ ID NO: 25 - DNA, 20 bp, Artificial Sequence
  synthetic scrambled oligomer
ctcgatctcactctcgcgac

(19) United States
(12) Patent Application Publication
(10) Pub. No.: US 2005/0153935 A1
(43) Pub. Date: Jul. 14, 2005

(54) Compound and method for treating androgen-independent prostate cancer

(76) Inventors: Patrick L. Iversen, Corvallis, OR (US); Gayathri R. Devi, Morrisville, NC (US)

Correspondence Address: PERKINS COIE LLP, P.O. BOX 2168, MENLO PARK, CA, 94026, US

(21) Appl. No.: 10/938,171

(22) Filed: Sep. 10, 2004

Related U.S. Application Data
(60) Provisional application No. 60/502,343, filed on Sep. 12, 2003.

(51) Int. Cl. A61K 48/00

(52) U.S. Cl. 514/80

(57) ABSTRACT

The present invention provides compositions and methods for treating prostate cancer. The composition comprises a morpholino antisense compound having uncharged phosphorus-containing backbone linkages and a base sequence that is complementary to a target region containing at least 12 contiguous bases in a preprocessed or processed human androgen receptor transcript. The method is designed for treating prostate cancer in a subject having a hormone-refractory (androgen-independent) prostate cancer.

This application claims the benefit of U.S. Provisional Application No. 60/502,343 filed Sep. 12, 2003, which is incorporated herein by reference in its entirety.

### FIELD OF THE INVENTION

This invention relates to antisense oligomers for use in treating prostate cancer in humans, anticancer treatment methods employing the oligomers, and methods for monitoring efficacy of antisense oligomers in prostate cancer therapy.

### BACKGROUND OF THE INVENTION

Prostate cancer is the second leading cause of cancer related mortality in the United States. In 2001, there were 198,100 new cases and 31,500 deaths reported from prostate cancer. Routine testing for increased levels of prostate specific antigen (PSA) in men past the age of 50 has increased detection of early-stage, localized prostate cancer in men.

A number of therapies are available when localized prostate cancer is detected. These include hormone therapy to block or reduce androgen-androgen receptor interaction, prostatectomy, external beam radiation therapy and brachytherapy.

For more advance-stage metastatic prostate cancer, surgical or medical castration may be recommended, to eliminate testosterone produced by the testes (androgen ablation monotherapy). Some patients are also treated with a direct androgen receptor antagonist (flutamide or bicalutamide in the United States) in an effort to block residual androgens which are produced outside the testes (primarily by the adrenals) and converted into testosterone and dihydrotestosterone.

Most patients respond to androgen ablation therapy, but the majority relapse within 2-3 years and virtually all relapse within 5-7 years. These recurrent tumors appear clinically to be androgen independent, as evidence by a lack of response of PSA levels to androgen-suppression therapy, even though androgen receptor is expressed by virtually all androgen-independent prostate cancers, possibly even at increased levels relative to the primary tumors in most cases (Taplin, M. E., et al., Cancer Res. 59: 2511-2515 (1999); Hobisch, A., et al., Cancer Res. 55: 3068-3072 (1995)). A prostate cancer that has progressed to an androgen-independent stage is typically refractory to therapies used to treat androgen-dependent prostate cancers.

Treating prostate tumors at this more refractory stage represents a major challenge in prostate-tumor therapy, and there is thus a need for useful treatments for more advanced-stage forms of prostate cancer that have progressed to an androgen-independent stage.

### SUMMARY OF THE INVENTION

In one aspect, the invention includes an oligonucleotide analog compound for use in method of treating prostate cancer in a subject. The compound is characterized by: (i) 12-40 morpholino subunits, (ii) a substantially uncharged, phosphorus-containing backbone linking the subunits, (iii) active uptake by human prostate cancer cells, (iv) a base sequence that is complementary to a target region containing at least 12 contiguous bases in a preprocessed or processed human androgen receptor transcript, and which includes at least 6 contiguous bases of the sequence selected from the group consisting of: SEQ ID NOS:2, 7, 8, and 9-22, and (v) capable of hybridizing with a preprocessed human androgen-receptor transcript to form a heteroduplex structure having a Tm of dissociation of at least 45° C.

The treatment compound may be composed of morpholino subunits linked by uncharged, phosphorus-containing intersubunit linkages joining a morpholino nitrogen of one subunit to a 5′ exocyclic carbon of an adjacent subunit. The intersubunit linkages in the compound may be phosphorodiamidate linkages, preferably in accordance with the structure: 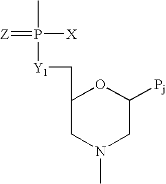
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino. In an exemplary compound, X=NR₂, where each R is independently hydrogen or methyl in the compound administered.

In one general embodiment, the compound administered is effective to target the start site of the processed human androgen transcript, and has a base sequence that is complementary to a target region containing at least 12 contiguous bases in a processed human androgen receptor transcript, and which includes at least 6 contiguous bases of one of the sequences SEQ ID NOS:2, 7, 8.

The compound may include a base sequence having one of these sequences.

In another general embodiment, the compound administered is effective to target a splice site of preprocessed human androgen transcript and has a base sequence that is complementary to a target region containing at least 12 contiguous bases in a processed human androgen receptor transcript, and which includes at least 6 contiguous bases of one of the sequences SEQ ID NOS:9-22.

The compound may also include a non-oligomeric chemotherapeutic agent. The compound may be used to treat hormone-responsive (androgen-dependent) or hormone-refractory (androgen-independent) prostate cancer.

In another aspect, the invention includes a method of treating prostate cancer in a subject having an androgen-independent prostate cancer, as evidenced by a lack of response in PSA level to androgen-suppression therapy. In practicing the method, the subject is given an oligonucleotide analog compound of the type described above. Following administration of the compound to the patient, the subject's serum PSA level is monitored, and compound administration is continued, on a periodic basis, at least until a substantial drop in the subject's serum PSA level is observed.

The method may further include administering a chemotherapeutic agent to the subject. The method may also include, at a selected time after administering the compound, obtaining a sample of a body fluid from the subject; and assaying the sample for the presence of a nuclease-resistant heteroduplex composed of an oligonucleotide analog compound complexed with a complementary portion of a preprocessed human androgen receptor transcript.

In still another aspect, the invention includes a method of confirming the presence of an effective interaction between a human androgen-receptor pre-processed transcript and an uncharged morpholino oligonucleotide analog compound. In practicing the method, the subject is administered a compound characterized by: (i) 12-40 morpholino subunits, (ii) a substantially uncharged, phosphorus-containing backbone linking said subunits, (iii) active uptake by human prostate cancer cells, (iv) a base sequence that is complementary to a target region containing at least 12 contiguous bases in a preprocessed human androgen receptor transcript, and (v) capable of hybridizing with a preprocessed human androgen-receptor transcript to form a heteroduplex structure having a Tm of dissociation of at least 45° C.

At a selected time after this administering, a sample of a body fluid from the subject is obtained and assayed for the presence of a nuclease-resistant heteroduplex composed of the oligonucleotide analog compound complexed with a complementary-sequence portion of a preprocessed human androgen-receptor transcript.

These and other objects and features of the invention will become more fully apparent when the following detailed description of the invention is read in conjunction with the accompanying drawings.

### BRIEF DESCRIPTION OF THE FIGURES

FIG. 1A shows antisense androgen receptor PMO specificity in an in vitro androgen receptor-luciferase hybrid gene (pCiNeo AR-LucΔA) plasmid-based test system. FIG. 1A demonstrates inhibition of translation in a rabbit reticulocyte lysate with various concentrations of vehicle (water), antisense or scrambled PMOs in the presence of the reporter gene RNA.

FIG. 1B depicts HeLa cells transiently transfected with pCiNeo AR-LucΔA after treatment with vehicle, antisense or scrambled PMOs at the indicated concentrations.

FIG. 1C is an immunoblot showing the effect of androgen receptor antisense PMO on androgen receptor protein expression in LNCaP androgen-dependent human prostate cells.

FIG. 2 shows the effect on serum PSA levels in vivo in mice bearing androgen dependent LAPC-4 human prostate cancer xenografts pre and post-treatment with an antisense androgen receptor PMO and a scrambled control PMO.

FIG. 3 shows an immunoblot of in vivo androgen receptor levels from the same mouse xenograft system as described for FIG. 2. The samples are from three mice pre and post-treatment with antisense androgen receptor PMOs.

FIG. 4 depicts the effect of antisense androgen receptor PMO on androgen independent LAPC-4 human xenografts. The immunoblot shows androgen receptor levels from two mice pre-treatment and after a 14-day treatment with PMO.

FIGS. 5A-5B show immunohistochemical evidence of: a decrease in nuclear androgen receptor staining of LAPC4 androgen independent tumors following extended treatment with human androgen receptor antisense oligomers (FIG. 5A); nuclear androgen staining in LAPC4 androgen independent xenograft prior to androgen receptor antisense administration (FIG. 5B). Magnification was at 50× under oil immersion.

FIG. 6 shows the down-regulation of androgen receptor protein in normal mouse prostate after intraperitoneal treatment with antisense androgen receptor PMO. The indicated amount of PMO was injected daily for four days and the ventral prostates were harvested on day five and analyzed for androgen receptor by immunoblotting.

FIGS. 7A-7B show the effect of antisense human (hAR) or mouse (mAR) androgen receptor PMO (200-800 μg/day) on mouse prostate after administration to normal male ICR mice. The immunoblot (FIG. 7A) demonstrates a dose dependent reduction of the androgen receptor using either mAR or hAR PMO compared to the level of androgen receptor expression in the prostate of saline or scrambled PMO treated mice. The immunoblot was stripped and reprobed to determine the beta-actin levels which act as internal standards. A graph of the ratio of AR to actin is shown in FIG. 7B.

FIG. 8 contains representative HPLC chromatograms showing detection of the androgen receptor antisense oligomer in tissue lysates from; (A) untreated plasma; (B) untreated liver; (C-F) tissue lysates (as indicated) from LAPC4 xenograft mice 24 hours following intraperitoneal administration of 400 μg of androgen receptor antisense PMO.

FIGS. 9A-E show several preferred morpholino subunits having 5-atom (FIG. 9A), six-atom (FIG. 9B) and seven-atom (FIGS. 9C-9E) linking groups suitable for forming polymers.

FIGS. 10A through 10E show the repeating subunit segment of exemplary morpholino oligonucleotides, constructed using the subunits depicted in FIGS. 9A-9E, respectively.

### DETAILED DESCRIPTION OF THE INVENTION

I. Definitions

The androgen receptor (AR) gene is a member of the steroid/nuclear receptor gene superfamily. The DNA sequence for the human androgen receptor is available as GenBank Accession numbers M35845 and M35846.

Prostate specific antigen (PSA) is a glycoprotein produced by the cells of the prostate gland, primarily by the epithelial cells that line the acini and ducts of the prostate gland. PSA is concentrated in prostatic tissue, and serum PSA levels are normally very low. Elevated levels of serum PSA are associated with prostate pathologies including prostate cancer.

The terms “antisense oligonucleotide” and “antisense oligomer” are used interchangeably and refer to a sequence of nucleotide bases and a subunit-to-subunit backbone that allows the antisense oligomer to hybridize to a target sequence in an RNA by Watson-Crick base pairing, to form an RNA:oligomer heteroduplex within the target sequence. The antisense oligonucleotide includes a sequence of purine and pyrimidine heterocyclic bases, supported by a backbone, which are effective to hydrogen-bond to corresponding, contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in length, linked together by phosphorous-containing linkages one to three atoms long.

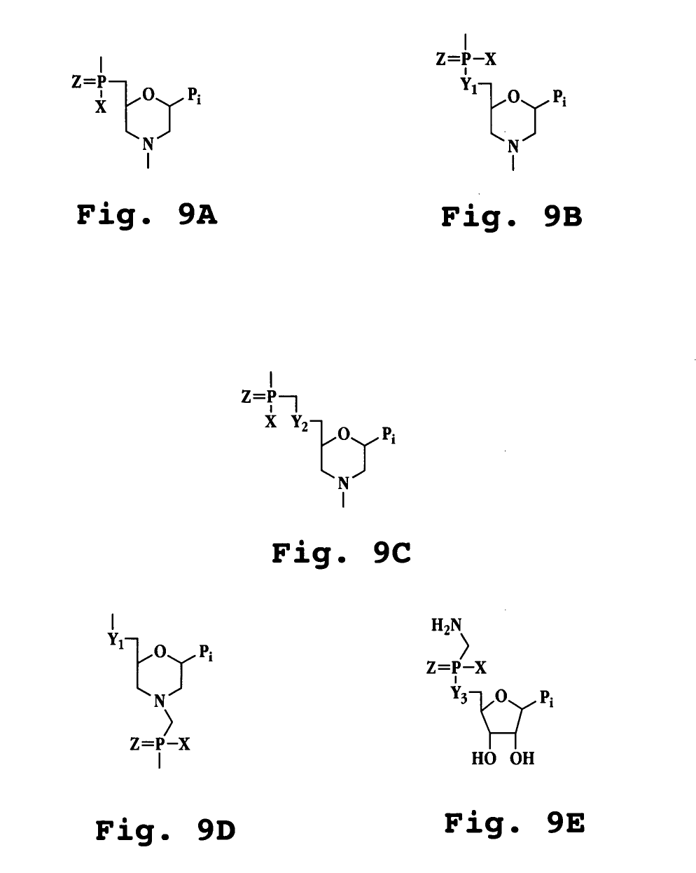

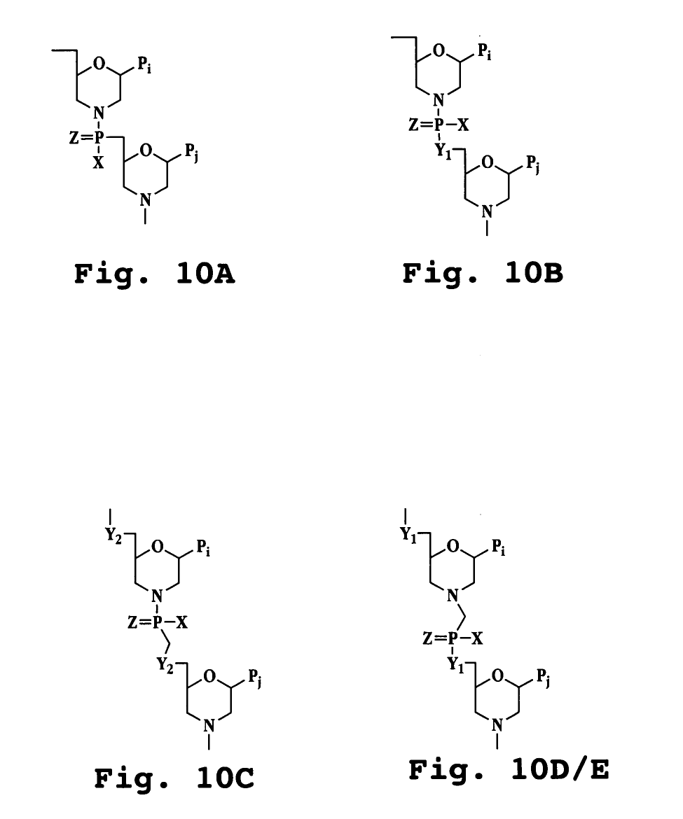

A “morpholino” oligonucleotide refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. A preferred “morpholino” oligonucleotide is composed of morpholino subunit structures of the form shown in FIGS. 9A-9E, where (i) the structures are linked together by phosphorous-containing linkages, one to three atoms long, joining the morpholino nitrogen of one subunit to the 5′ exocyclic carbon of an adjacent subunit, and (ii) Pᵢ and Pⱼ are purine or pyrimidine base-pairing moieties effective to bind, by base-specific hydrogen bonding, to a base in a polynucleotide. Exemplary structures for antisense oligonucleotides for use in the invention include the morpholino subunit types shown in FIGS. 9A-E, with the linkages shown in FIGS. 10A-E.

As used herein, an oligonucleotide or antisense oligomer “specifically hybridizes” to a target polynucleotide if the oligomer hybridizes to the target under physiological conditions, with a thermal melting point (Tm) substantially greater than 37° C., preferably at least 45° C., and typically 50° C.-80° C. or higher. Such hybridization preferably corresponds to stringent hybridization conditions, selected to be about 10° C., and preferably about 50° C. lower than the Tm for the specific sequence at a defined ionic strength and pH. At a given ionic strength and pH, the Tm is the temperature at which 50% of a target sequence hybridizes to a complementary polynucleotide.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules.

As used herein the term “analog” with reference to an oligomer means a substance possessing both structural and chemical properties similar to those of the reference oligomer.

As used herein, a first sequence is an “antisense sequence” with respect to a second sequence if a polynucleotide whose sequence is the first sequence specifically binds to, or specifically hybridizes with, the second polynucleotide sequence under physiological conditions.

As used herein, the term “androgen receptor antisense compound” refers to an antisense morpholino compound having high affinity (i.e., “specifically hybridizes”) to a complementary or near-complementary the androgen receptor nucleic acid sequence, e.g., the sequence including and spanning the normal AUG start site.

As used herein the term “analog” in reference to an oligomer means a substance possessing both structural and chemical properties similar to those of the reference oligomer.

As used herein, “effective amount” relative to an antisense oligomer refers to the amount of antisense oligomer administered to a subject, either as a single dose or as part of a series of doses, that is effective to inhibit expression of a selected target nucleic acid sequence.

Abbreviations:
PMO=morpholino oligomer
AR=androgen receptor
PSA=prostate specific antigen
   II. Antisense Compound

The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, all of which are incorporated herein by reference.

The antisense oligomers (compounds) of the present invention are composed of morpholino subunits of the form shown in the above cited patents, where (i) the morpholino groups are linked together by uncharged phosphorus-containing linkages, one to three atoms long, joining the morpholino nitrogen of one subunit to the 5′ exocyclic carbon of an adjacent subunit, and (ii) the base attached to the morpholino group is a purine or pyrimidine base-pairing moiety effective to bind, by base-specific hydrogen bonding, to a base in a polynucleotide. The purine or pyrimidine base-pairing moiety is typically adenine, cytosine, guanine, uracil or thymine. Preparation of such oligomers is described in detail in U.S. Pat. No. 5,185,444 (Summerton and Weller, 1993), which is hereby incorporated by reference in its entirety. As shown in the reference, several types of nonionic linkages may be used to construct a morpholino backbone.

Exemplary backbone structures for antisense oligonucleotides of the invention include the β-morpholino subunit types shown in FIGS. 9A-9E. It will be appreciated that a polynucleotide may contain more than one linkage type.

The subunit shown in FIG. 9A contains a 1-atom phosphorous-containing linkage which forms the five atom repeating-unit backbone shown in FIG. 10A, where the morpholino rings are linked by a 1-atom phosphoamide linkage.

The subunit shown in FIG. 9B is designed for 6-atom repeating-unit backbones, as shown in FIG. 10B. In the subunit structure, the atom Y linking the 5′ morpholino carbon to the phosphorous group may be sulfur, nitrogen, carbon or, preferably, oxygen. The X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures.

The subunits shown in FIGS. 9C-9E are designed for 7-atom unit-length backbones as shown in FIGS. 10C-10E, respectively. In the structure shown in FIG. 9C, the X moiety is as in FIG. 9B and the moiety Y may be a methylene, sulfur, or preferably oxygen. In the structure shown in FIG. 9D the X and Y moieties are as in FIG. 9B. In the structure seen in FIG. 9E, X is as in FIGS. 9B and Y is O, S, or NR. In all subunits depicted in FIGS. 9A-9E, Z is O or S, and Pᵢ or Pⱼ is adenine, cytosine, guanine or uracil.

The processing of nuclear RNA following transcription is observed in virtually all living cells. The mammalian genome contains genes that make transcripts of approximately 16,000 bases in length containing 7 to 8 exons. The process of splicing reduces the length of the mRNA to an average of 2,200 bases. The initial transcript is referred to as heterologous nuclear RNA (hnRNA) or pre-mRNA. Processing of hnRNA involves an aggregate of approximately 20 proteins, referred to collectively as the spliceosome, which carries out splicing and transport of mRNA from the nucleus. The spliceosome does not appear to scan from a common direction for all transcripts; introns may be removed in a reproducible order but not in a directional order. For example, introns 3 and 4 may be removed first, followed by removal of introris 2 and 5, followed by removal of introns 1 and 6. The order of intron removal is not predictable a priori of observation. The sequence recognition for processing is small, suggesting that errors or multiplicity of processing sites can be anticipated, and, in fact, as more genes are investigated, more variation in processing of hnRNA has been observed.

In preprocessed mRNA, the two-base sequence motifs at exon/intron junctions are invariant. The upstream (5′) splice donor (SD) junction is of the form exon-/GT-intron, while the downstream (3′) splice acceptor (SA) junction is of the form intron-AG/exon. The flanking bases are not invariant; however, the base immediately upstream of the splice acceptor AG sequence is C about 80% of the time.

The region of the mRNA against which the compound is directed also referred to herein as the target sequence. The AR mRNA to which the antisense binds may be preprocessed (prespliced) mRNA, in which case the antisense compound may act to interfere with correct splicing, leading to truncated forms of the translated protein, or may bind to the processed mRNA, leading to inhibition of translation. The compound has a base sequence that is complementary to a target region containing at least 12 contiguous bases in a preprocessed or processed human androgen receptor transcript. The compound sequence preferably includes at least six contiguous bases of one of the sequences identified as SEQ ID NO:2-5 and 7-22. Preferably, the compound is capable of hybridizing with the target sequence to form a heteroduplex structure having a Tm of dissociation of at least 45° C.

In one embodiment, the compound has a sequence which spans the start codon of the androgen receptor mRNA, meaning the compound contains a sequence complementary to a region of AR RNA containing the AUG mRNA start site and adjacent 5′ and 3′ base(s). In this embodiment, the compound preferably contains an internal 3-base triplet complementary to the AUG site, and bases complementary to one or more bases 5′ and 3′ to the start site. Preferably, the antisense oligomer is complementary to a target region of a selected processed mRNA coding for the androgen receptor protein. Exemplary antisense oligomers that are targeted to the start site of the androgen receptor are given in Table 1.

TABLE 1
Exemplary Antisense Oligomers Targeting the Start Site of
Processed Androgen Receptor Transcript

| | | | SEQ. |
|---|---|---|---|
| Targeted | | | ID |
| Region | Antisense Oligomer (5′ to 3′) | Species | NO. |
| −8 to +12 | CTGCACCTCCATCCTTGAGC | Mouse | 1 |
| −8 to +12 | CTGCACTTCCATCCTTGAGC | Human | 2 |
| −8 to +10 | GCACTTCCATCCTTGAGC | Human | 3 |
| −7 to +10 | GCACTTCCATCCTTGAG | Human | 4 |
| −11 to +12 | CTGCACTTCCATCCTTGAGCTTC | Human | 5 |
| −11 to −31 | GTCTGTAGCTTCCACCGAATT | Mouse | 6 |
| −11 to −31 | GGCTGAATCTTCCACCTACTT | Human | 7 |
| +4 to +23 | CCTTCCCAGCCCTAACTGAC | Mouse & | 8 |
| | | Human | |

Targeted regions are relative to the AUG codon. The oligomer sequences show the antisense of the AUG start codon (CAT) in bold when included. The above sequences were derived from GenBank Accession numbers X59592 for mouse and M21748 for human.

In another embodiment, the compound has a sequence which spans the splice acceptor junction of nuclear (unspliced) RNA. This compound is RNase-inactive, that is, does not promote cleavage of bound RNA and is believed to act by sterically blocking the molecular machinery from transcribing, processing, or translating the target sequence. In yet another embodiment, the compounds target a sequence downstream of the splice acceptor junction, i.e. within the exon. In a preferred embodiment, the antisense oligomer is complementary to a target region of a selected preprocessed mRNA coding for a selected protein, where the 5′ end of the target region is 1 to 25 bases downstream, preferably 2 to 20 bases downstream, and more preferably 2 to 15 bases downstream, of a normal splice acceptor site in the preprocessed mRNA. Thus, the antisense oligomer is effective to inhibit splicing at the normal splice acceptor site and thus produce splice variant mRNA, leading to truncated or otherwise aberrant versions of the selected protein upon translation. Exemplary antisense oligomers that are targeted to the androgen receptor splice site of the androgen receptor are given in Table 2. In a preferred embodiment, the compound includes a base sequence selected from the group consisting of SEQ ID NO:9-22.

TABLE 2
Exemplary Antisense Oligomers Targeting a Splice Site of
Preprocessed Human Androgen Receptor Transcript

| | | | | SEQ. |
|---|---|---|---|---|
| | Targeted | | Genbank | ID |
| PMO | Ncts. | Antisense Oligomer (5′ to 3′) | Acc. No. | NO. |
| Ex1SD | 1676-1696 | 5′-CTTACCGCATGTCCCCGTAAG-3′ | M27423 | 9 |
| Ex2SA | 81-99 | 5′-CTCCAAACTGGAAAGACAC-3′ | M27424 | 10 |
| Ex2SD | 235-254 | 5′-GACCCTTTACCTTCAGCGGC-3′ | M27424 | 11 |
| Ex3SA | 133-152 | 5′-GGTACTTCTGTTTCCCTGGG-3′ | M27425 | 12 |
| Ex3SD | 244-263 | 5′-GTATCTTACCTCCCAGAGTC-3′ | M27425 | 13 |
| Ex4SA | 121-139 | 5′-CAGCTTCCGGGCTATTGGG-3′ | M27426 | 14 |
| Ex4SD | 406-428 | 5′-CCTTTTCCTTACCAGGCAAGGCC-3′ | M27426 | 15 |
| Ex5SA | 36-56 | 5′-GGAAGCCTGGAGAAGAAGAGG-3′ | M27427 | 16 |
| Ex5SD | 184-203 | 5′-GCACTTACTCATTGAAAACC-3′ | M27427 | 17 |
| Ex6SA | 52-71 | 5′-GCATGCGGTACCTGGGAAGG-3′ | M27428 | 18 |
| Ex6SD | 181-200 | 5′-GGCACTTACTAATGCTGAAG-3′ | M27428 | 19 |
| Ex7SA | 200-218 | 5′-CCACTGGAACTGATGTGGG-3′ | M27429 | 20 |
| Ex7SD | 359-378 | 5′-CGTTTGCTTACAGGCTGCAC-3′ | M27429 | 21 |
| Ex8SA | 43-60 | 5′-CTCGCAATCTGTAGGGAAG-3′ | M27430 | 22 |

The compound is designed to hybridize under physiological conditions with a Tm greater than 45° C. Although the compound is not necessarily 100% complementary to the target sequence, it is effective to stably and specifically bind to the target sequence such that expression of the target sequence is modulated. The appropriate length of the oligomer to allow stable, effective binding combined with good specificity is about 8 to 40 nucleotide base units, and preferably about 12-25 base units. Mismatches, if present, are less destabilizing toward the end regions of the hybrid duplex than in the middle. Oligomer bases that allow degenerate base pairing with target bases are also contemplated, assuming base-pair specificity with the target is maintained.

The solubility of the antisense compound, and the ability of the compound to resist precipitation on storage in solution, can be further enhanced by derivatizing the oligomer with a solubilizing moiety, such as a hydrophilic oligomer, or a charged moiety, such as a charged amino acid or organic acid. The moiety may be any biocompatible hydrophilic or charged moiety that can be coupled to the antisense compound and that does not interfere with compound binding to the target sequence. The moiety can be chemically attached to the antisense compound, e.g., at its 5′ end, by well-known derivatization methods. One preferred moiety is a defined length oligo ethylene glycol moiety, such as triethyleneglycol, coupled covalently to the 5′ end of the antisense compound through a carbonate linkage, via a piperazine linking group forming a carbamate linkage with triethyleneglycol, where the second piperazine nitrogen is coupled to the 5′-end phosphorodiamidate linkage of the antisense. Alternatively, or in addition, the compound may be designed to include one a small number of charged backbone linkages, such as a phosphodiester linkage, preferably near one of the ends of the compound. The added moiety is preferably effective to enhance solubility of the compound to at least about 30 mg/ml, preferably at least 50 mg/ml in aqueous medium.

Additional sequences may be prepared by one of skill in the art, having in mind one or more desired target sequences, with screening carried out according to methods routinely employed by those of skill in the art.

III. Treatment Methods

In accordance with another aspect of the invention, the compound above is used in the treatment of androgen independent prostate cancer by inhibiting or altering expression of the androgen receptor.

The method is carried out by administering to the subject an antisense oligomer characterized by 12-40 morpholino subunits and having a substantially uncharged, phosphorus-containing backbone linking said subunits. The oligomer has a base sequence that is complementary to a target region containing at least 12 contiguous bases in a preprocessed human androgen receptor transcript, and the oligomer is capable of hybridizing with a preprocessed human androgen-receptor transcript to form a heteroduplex structure having a Tm of dissociation of at least 45° C.

As seen in Tables 4 and 5, in vivo results show that human prostate cancer cells actively uptake the antisense oligomers of the invention. Mice bearing LAPC-4 tumors treated with a single dose of androgen receptor antisense PMO (400 or 800 μg) showed accumulation of the PMO in the tumor, prostate, liver and kidney.

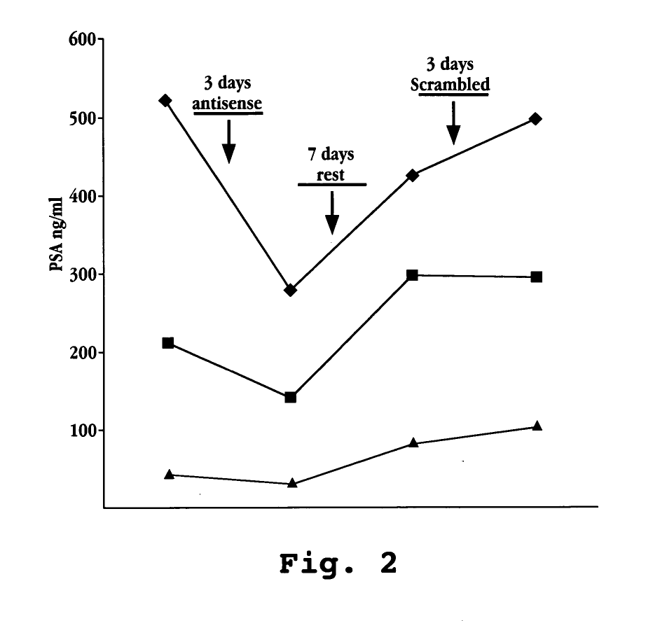

The in vivo effectiveness of the PMO on mice bearing androgen dependent LAPC-4 is seen in FIG. 2. Three mice were treated for three days with antisense compound, rested for seven days, and treated with a scrambled PMO for three days. As seen in FIG. 2, all three results showed a decrease in serum PSA (ng/ml). Further, all three showed an increase in serum PSA level with the scrambled PMO.

A. In Vivo Administration Of Antisense Oligomers.

Effective delivery of the antisense oligomer to the target is an important aspect of the methods of the invention. In accordance with the invention, such routes of antisense oligomer delivery include, but are not limited to, various systemic routes, including oral and parenteral routes, e.g., intravenous, subcutaneous, intraperitoneal, and intramuscular, as well as inhalation and transdermal delivery. It will be appreciated that any methods which are effective to deliver the antisense oligomer to the target cells or to introduce the drug into the bloodstream are contemplated.

Therapeutic compositions for injection or infusion may take such forms as suspensions, solutions or emulsions of the antibody in oily or aqueous vehicles, and, may contain components such as suspending, stabilizing and/or dispensing agents. Alternatively, the composition may be in a dry form, for reconstitution before use with an appropriate sterile liquid.

Parenteral administration includes injection or gradual infusion over time. The compounds of the invention can be injected intravenously, intraperitoneally, intramuscularly, subcutaneously, intratumorally; or administered transdermally or by peristaltic means. In a preferred embodiment, the compound is administered intraperitoneally. Suitable regimens for administration are variable, but are typified by an initial administration followed by repeated doses at one or more intervals by subsequent administration.

Transdermal delivery of antisense oligomers may be accomplished by use of a pharmaceutically acceptable carrier adapted for e.g., topical administration. One example of morpholino oligomer delivery is described in PCT patent application WO 97/40854, incorporated herein by reference.

The antisense oligomer may be administered directly to a subject or in a suitable pharmaceutical carrier. In one embodiment, at least one antisense compound is administered with a physiologically acceptable carrier, excipient, or diluent, where the antisense compound is dissolved or dispersed therein as an active ingredient and formulated according to conventional practice. The carrier may be any of a variety of standard physiologically acceptable carrier employed by those of ordinary skill in the art. Examples of such pharmaceutical carriers include, but are not limited to, saline, phosphate buffered saline (PBS), water, aqueous ethanol, emulsions such as oil/water emulsions, triglyceride emulsions, wetting agents, tablets and capsules. It will be understood that the choice of suitable physiologically acceptable carrier will vary dependent upon the chosen mode of administration.

In some instances liposomes may be employed to facilitate uptake of the antisense oligonucleotide into cells. (See, e.g., Williams, 1996; Lappalainen, et al., 1994; Uhlmann, et al., 1990; Gregoriadis, 1979.) Hydrogels may also be used as vehicles for antisense oligomer administration, for example, as described in WO 93/01286. Alternatively, the oligonucleotides may be administered in microspheres or microparticles. (See, e.g., Wu and Wu, 1987).

Sustained release compositions are also contemplated within the scope of this application. These may include semipermeable polymeric matrices in the form of shaped articles such as films or microcapsules.

As described above, the compound may also include or be administered in combination with a moiety that enhances the solubility of the compound. The moiety preferably enhances the solubility in aqueous medium to between 25-50 mg/ml or greater. A preferred moiety is a polyethylene glycol (PEG) chain.

In one embodiment, the antisense oligomer may be administered at regular intervals for a short time period, e.g., daily for two weeks or less. In another embodiment, the antisense oligomer is administered intermittently over a longer period of time. It will be appreciated that antisense oligomer administration may be continued for an indefinite time period. Typically, one or more doses of antisense oligomer are administered. Preferred doses for oral administration are from about 1 mg oligomer/patient to about 25 mg oligomer/patient (based on an adult weight of 70 kg). In some cases, doses of greater than 25 mg oligomer/patient may be necessary. For IV administration, the preferred doses are from about 0.5 mg oligomer/patient to about 10 mg oligomer/patient (based on an adult weight of 70 kg). Dosages will vary in accordance with such factors as the age, health, sex, size and weight of the patient, the route of administration, and the efficacy of the oligonucleotide agent with respect to the particular disease state. Greater or lesser amounts of oligonucleotide may be administered as required.

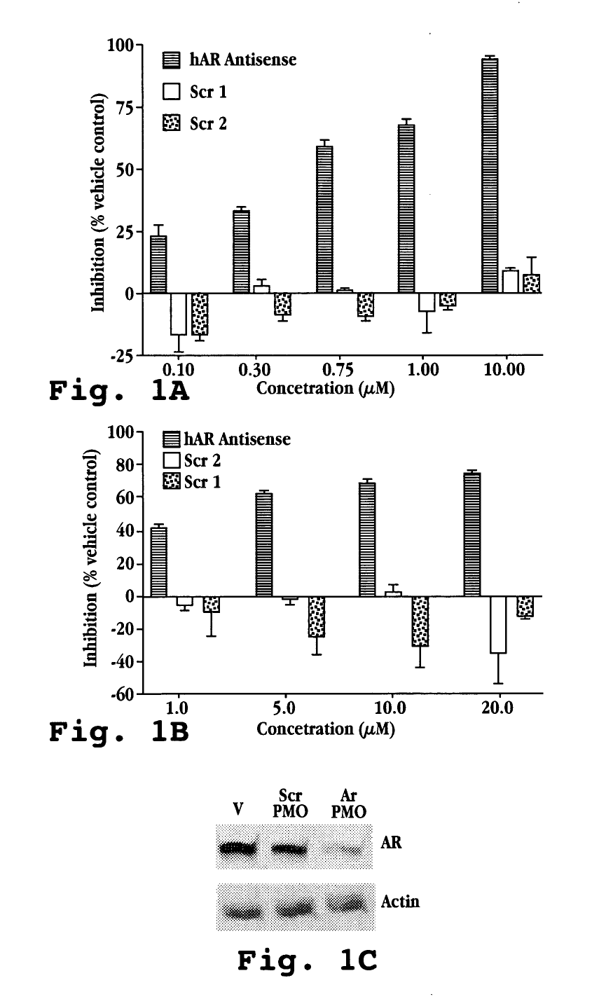

Inhibition of the androgen receptor is dose dependent. FIGS. 1A-1B show a graph of inhibition of luciferase activity by percentage of vehicle control by concentration of human androgen receptor PMO (SEQ ID NO:2). As seen in FIG. 1A, this inhibition was sequence-specific as the scrambled PMO controls showed no such effect. In FIG. 1B, scrape loaded delivery of the androgen receptor antisense PMO in the HeLa cells expressing the androgen receptor-luciferase protein caused a dose-dependent decrease in luciferase activity. A corresponding decrease was not observed with the scrambled PMOs or vehicle control. Specific reduction of androgen receptor levels after treatment with 100 μM androgen receptor antisense PMO in the LNCaP cells compared to treatment with similar concentrations of the scrambled PMO control (FIG. 1C).

To determine whether the androgen receptor antisense PMO could inhibit expression of endogenous full-length androgen receptor transcripts, they were introduced into the androgen receptor expressing and androgen-responsive LNCaP cell line. PMO delivery by syringe loading technique in a dose range of 50-200 uM has been found to be optimal for LNCaP cells in culture (Devi, G. R., et al., Prostate 53(3): 200-210 (2002)). Equal amounts of cell lysate protein prepared 24 h post PMO treatment were run on an electrophoresis gel and probed with androgen receptor specific antibody. The data presented in FIG. 1C shows specific reduction of androgen receptor levels after treatment with 100 μM androgen receptor antisense PMO in the LNCaP cells compared to treatment with similar concentrations of the scrambled PMO control. The immunoblot was stripped and reprobed to determine the beta-actin levels which act as internal standards.

It will be understood that the effective in vivo dose of the antisense oligonucleotides for use in the methods of the invention will vary according to the frequency and route of administration as well as the condition of the subject under treatment. Accordingly, such in vivo therapy will generally require monitoring by tests appropriate to the condition being treated as described further below. Adjustment in the dose or treatment regimen corresponding to the results of such monitoring may be used in order to achieve an optimal therapeutic outcome.

An effective in vivo treatment regimen using the antisense oligonucleotides of the invention will vary according to the frequency and route of administration, as well as the condition of the subject under treatment. Optimum dosages for a given route can be determined by routine experimentation according to methods known in the art. Such in vivo therapy is generally monitored by tests appropriate to the particular type of ailment being treated, and a corresponding adjustment in the dose or treatment regimen can be made in order to achieve an optimal therapeutic outcome.

It will further be appreciated that the use of an antisense oligonucleotide to treat prostate cancer may be used following, concurrently with and/or prior to additional therapeutic intervention, including, but not limited to, radical prostatectomy, radiation therapy, and chemotherapy.

B. Monitoring Treatment

Effective delivery of the antisense oligomer to the target mRNA is an important aspect of the method. PMOs have been shown to enter cells efficiently (see e.g. Summerton, et al., Antisense Nucleic Acid Drug Dev. 7: 63-70, (1997), and copending and co-owned U.S. patent application Ser. No. 09/493,427).

The efficacy of a given therapeutic regimen involving the methods described herein, may be monitored by one or more of (1) histology or immunohistology, by staining prostate cells or tissue sections to evaluate the status of the prostate tumor; (2) analysis of tissue lysates for the presence of androgen receptor PMO; (3) a determination of serum prostate specific antigen (PSA), as an indicator of prostate pathology; (4) monitoring the presence or absence in a cell culture of the encoded, full length protein as determined by ELISA or Western blotting, and (5) detection of tumor size by ultrasound, computed tomography (CT) or magnetic resonance imaging (MRI). Numerous example of such methods are generally known in the art, some of which are further described below.

Additionally, a morpholino antisense compound of the type disclosed herein, when administered in vivo, can be detected in the urine of the receiving subject in a heteroduplex form consisting of the antisense compound and its RNA complement. This verifies that the antisense compound has been taken up by the target tissue and allows the practitioner to monitor the effectiveness of the treatment method, e.g. the effectiveness of various modes of administration, and dosages giving maximal or near-maximal levels of heteroduplex in the urine.

In a preferred embodiment, the effectiveness of the AR antisense sequence is determined by monitoring serum levels of PSA. Serum level of PSA is determined by ELISA. When the prostate gland enlarges, due to cancer or benign conditions, PSA levels in the blood tend to rise. The PSA level that is considered normal for an average man ranges from 0 to 4 (ng/ml). A PSA level of 4 to 10 ng/ml is considered slightly elevated; levels between 10 and 20 ng/ml are considered moderately elevated; and levels above 20 ng/ml are considered highly elevated.

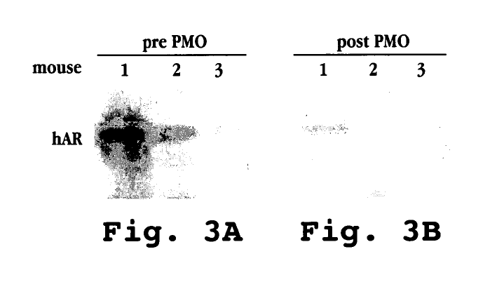

Treatment with antisense PMO decreased on serum PSA levels in vivo as seen in FIG. 3. Three mice (lanes 1-3) bearing androgen dependent LAPC-4 human prostate cancer xenografts were treated for three days with 200 μg PMO (i.p.). As seen post experiment, PSA levels decrease, indicating a decrease in prostate pathology.

The antisense oligomer treatment regimen may be adjusted (dose, frequency, route, etc.), as indicated, based on the results of the various assays described above.

Materials and Methods

Oligomer synthesis. Phosphorodiamidate morpholino oligomers (PMOs) were synthesized at AVI BioPharma Inc. (Corvallis, Oreg.) as previously described (Summerton, J., Biochim. Biophys. Acta. 1489(1): 141-158 (1999)). Purity was >95% as determined by reverse phase HPLC and MALDI TOF mass spectroscopy. The base compositions and sequences of the oligomers are shown in Table 1. The PMOs are aqueous soluble and were dissolved in sterile water for in vitro experiments and in saline for in vivo injections.

Plasmid-based test system for screening PMO antisense activity. A fusion construct was generated by subcloning 29 bases of the 5′ untranslated region, AUG translation start site, and by the first 16 bases of the protein coding sequence of androgen receptor gene followed by luciferase into the pCiNeo expression vector (Promega, Madison, Wis.). The AUG start site of luciferase was subjected to in vitro site-directed mutagenesis resulting in a single start site in the androgen receptor leader. This fusion construct was named pCiNeoAR-LucΔA. This plasmid features a T7 promoter capable of generating in vitro transcribed RNA from a cloned insert for use in the cell free rabbit reticulocyte in vitro translation reactions and a CMV promoter for constitutive expression in mammalian cells. In vitro transcription was carried out with T7 Mega script (Ambion, Austin, Tex.).

Cell Free Luciferase Assay. In vitro translation was performed by mixing rabbit reticulocyte lysate with known amounts of antisense, scrambled oligomers or vehicle (water) followed by addition of a known amount of the AR/Luc RNA (˜1 nM final conc.). The Promega luciferase assay reagent protocol was followed. The percent inhibition of luciferase activity compared to control was calculated based on readings from a luminometer (Cardinal, Santa Fe, N. Mex.).

Luciferase Assay in Cell culture. Confluent HeLa cells were transiently transfected with the pCiNeoAR-LucΔA plasmid using Lipofectamine (Gibco BRL) according to the manufacturer's directions. The cells were trypsinized 24 hours later and 6×10⁵ cells/well were plated in 6-well plates. The cells were allowed to adhere overnight and scrape loaded with vehicle or PMOs at different concentrations (Hudziak, R. M., et al., Antisense Nucleic Acid Drug Dev. 10(3): 163-176 (2000)). Cell lysates were prepared 24 h later, normalized for protein content and luciferase activity was determined using a luminometer.

Cell Culture. HeLa cells (ATCC, Rockville, Md.) were grown in DMEM/F12; LNCaP (ATCC, Rockville, Md.) in RPMI-1640 with 10% FBS and LAPC-4 cells (gift from C. Sawyers, UCLA) in IDDM with 10% FBS and 10 nM DHT. Media were supplemented with 100 U/ml penicillin and 75 U/ml streptomycin. FBS and the antibiotics were purchased from Life Technologies (Gathesburg, Md.).

PMO Delivery in Cells. The PMOs were delivered into LNCaP cells in culture by syringe loading as described (Devi, et al., 2002). Briefly, 1×10⁶ LNCaP cells/ml of growth medium were incubated for 20′ at 37° C. The desired amounts of PMO (100 μM) and PF-127 (2% w/v) were added and mixed. The cell suspension was drawn up in a sterile 1 ml syringe through a 25-⅝ gauge needle and then expelled by steady pressure on the plunger. The procedure was repeated four times. Growth medium (2 ml) was added to each sample and the cells were collected by centrifugation. The cell pellet was resuspended in 2 ml culture medium and plated in a 6-well plate.

PMO Treatment In Vivo. Male NCr nude mice were obtained at 5-6 weeks of age from Taconic (Germantown, N.Y.). Tumors were established by injecting 106 LAPC-4 cells mixed with Matrigel 1:1 into the flank of mice. To establish an androgen independent tumor, mice bearing LAPC-4 tumors were surgically castrated under general anesthesia. Following castration, the tumors were excised when growth was re-established (4-6 weeks). The tumors were minced and re-implanted with Matrigel in mice that had already been surgically castrated 7-10 days before.

HPLC Detection of PMO in Tissue Lysates. The tissue lysates of tumors and various organs from saline and androgen receptor antisense PMO treated groups were analyzed for the presence of androgen receptor PMO by HPLC analysis. A 10.0 μl aliquot (500 ng) of the internal standard PMO (15-mer; 5′- GAG GGG CAT CGT CGC-3′ SEQ ID NO:23) was added to each aliquot of tissue lysate sample (˜100 μL) contained in eppendorf tubes. A 300 μL aliquot of methanol was added to each sample and the tubes were vortexed. The tubes were centrifuged for 15 minutes using a high-speed centrifuge and the supernatants were transferred to new eppendorf tubes. A 100 μL aliquot of Tris buffer (pH 8) was added to each pellet and the tubes were vortexed again. The solution from each tube was removed using a pipet and combined with the supernatant from previous step. The combined supernatants were heated in a water bath at 70° C. for 15 minutes. Samples were re-centrifuged for 15 minutes and the supernatants were transferred to new eppendorf tubes. Samples were evaporated by placing the same tubes in speed-vac under vacuum for 10-12 hours. Each evaporated sample was reconstituted by adding a 200 μL aliquot of FDNA reagent mixture. The FDNA reagent mixture contained both the 5′-fluoresceinated DNA sequence complementary to analyte oligomer of interest and a 5′-fluoresceinated DNA sequence whose sequence was complementary to the internal standard PMO (5′FAM-GCG ACG ATG CCC CTC MC GT-3′ SEQ ID NO:24) at a concentration of 1.0 O.D. units/ml each. A set of analyte standards were prepared by spiking appropriate amounts of oligomer (10, 25, 50, 100, 250, 500& 1000 ng/l 00 μL) with the internal standard (500 ng). A set of quality control samples (250 ng/100 μL) were similarly prepared and analyzed. The samples were analyzed by injecting on to a Dionex DNA Pac PA-100 column (Dionex Corporation, Sunnyvale, Calif.) as described previously (Knapp, D. C., et al., Anticancer Drugs 14: 39-47 (2003)). The HPLC runs were monitored at excitation and emission wavelengths of 494 nm and 518 nm respectively. The standard curve was built using linear regression and the lysate samples were quantitated against the curve.

Tissue Androgen Receptor and PSA Analyses. Tumor biopsies were done under general anesthesia using sterile conditions. Samples were flash frozen and kept at −80° C. until analysis. Tumor slices were dissolved in 1% SDS heated to 75° C. and ground using an electric pestle. Equal amounts of protein extract were electrophoresed on 10% polyacrylamide Tris-SDS gels (BioRad, Hercules, Calif.) and then electrophorectically transferred to nitrocellulose for immunodetection. The membranes were then blocked in 5% nonfat dry milk in TBS with 0.2% Tween 20 for 1 hour at room temperature. The membranes were incubated overnight with a 1:1 mixture of two rabbit antibodies to the androgen receptor (C-19 and N-20 Santa Cruz Biotechnology, Santa Cruz, Calif.) at a dilution of 1:5000 in TBS with 0.2% Tween 20 and 5% nonfat milk followed by a 1 hour incubation with horseradish peroxidase conjugated anti-rabbit IgG (Promega, Madison, Wis.) at a dilution of 1:5000. Renaissance Western blot chemiluminescence reagent (LifeSciences, Boston, Mass.) was used to develop the membranes.

Androgen Receptor and PSA Immunohistochemistry. Tumor biopsies were preserved in paraffin blocks and sections were immunostained for androgen receptor using a rabbit anti-androgen receptor N-terminal antibody (PG-21, Upstate Biotechnology, Lake Placid, N.Y.) as described (Stanbrough, M., et al., Proc. Natl. Acad. Sci. U.S.A. 98: 10823-10828 (2001)) and PSA using a polyclonal goat IgG (C-19, Santa Cruz Biotechnology, Santa Cruz, Calif.).

Serum PSA Analysis. PSA analyses were conducted on blood collected by retro-orbital sinus bleeds under general anesthesia. Approximately 400-500 μl of blood in serum separator tubes was then immediately spun and separated. The serum was then kept at −20° C. and samples were run in batches for each experiment. PSA ELISA was performed on the MEIA Abbott AxSYM system.

### IV. EXAMPLES

### Example 1

### Specific Inhibition of Androgen Receptor Expression In Vitro by Antisense PMOs

In contrast to antisense oligonucleotides that act by a RNaseH mechanism, PMOs targeted to the AUG translational start site cause steric blockade of ribosomal assembly thus preventing protein translation. A plasmid-based test system was used for both cell-free and cellular screening of androgen receptor antisense PMO generated against the androgen receptor translational initiation site. A fusion construct, pCiNeoAR-LucΔA, was generated by subcloning a small segment of the human androgen receptor which includes the AUG translation start site followed in frame by the fire fly luciferase coding region into the pCiNeo expression vector (Promega, Madison, Wis.), which contains an upstream T7 RNA polymerase and CMV promoter. For in vitro studies, androgen receptor-luciferase mRNA (AR-LucΔA RNA) was generated using T7 RNA polymerase. This was added to a rabbit reticulocyte lysate in vitro translation mix containing antisense androgen receptor PMO, scrambled PMO (with the same base content, but random sequence), mismatch unrelated PMO or vehicle (water). The percent inhibition of luciferase activity in the presence of various concentrations of the PMOs, compared to the vehicle control, was calculated using a luminometer to measure luciferase activity. The results (FIG. 1A) show a dose-dependent inhibition of luciferase activity by antisense human androgen receptor PMO (SEQ ID NO:2). This inhibition was sequence-specific as the scrambled PMO controls showed no such effect.

The same construct (pCiNeoAR-LucΔA), when transiently transfected into HeLa cells also generated high levels of luciferase activity. Scrape loaded delivery of the androgen receptor antisense PMO in the HeLa cells expressing the androgen receptor-luciferase protein caused a dose-dependent decrease in luciferase activity, which was not observed with the scrambled PMOs or vehicle (FIG. 1B).

To determine whether the androgen receptor antisense PMO could inhibit expression of endogenous full-length androgen receptor transcripts, they were introduced into the androgen receptor expressing and androgen-responsive LNCaP cell line. PMO delivery by syringe loading technique in a dose range of 50-200 μM has been found to be optimal for LNCaP cells in culture (Devi, et al., 2002). Equal amounts of cell lysate protein prepared 24 h post PMO treatment were run on an electrophoresis gel and probed with androgen receptor specific antibody. The data presented in FIG. 1C shows specific reduction of androgen receptor levels after treatment with 100 μM androgen receptor antisense PMO in the LNCaP cells compared to treatment with similar concentrations of the scrambled PMO control. The immunoblot was stripped and re-probed to determine the beta-actin levels which act as internal standards.

### Example 2

### Effect of Androgen Receptor Antisense PMO on Androgen Sensitive Prostate Cancer Xenograft In Vivo

An antisense PMO based upon the human androgen receptor (AR) translational initiation site and a scrambled control PMO were synthesized and purified to greater than 95% as determined by reverse phase HPLC and MALDI TOF mass spectrometry. The sequences are shown below:

| | |
|---|---|
| Human AR PMO | |
| 5′-CTGCACTTCCATCCTTGAGC-3′ | (SEQ ID NO:2) |
| Scrambled PMO | |
| 5′-CTCGATCTCACTCTCGCGAC-3′ | (SEQ ID NO:25) |

These PMOs were tested in mice bearing the androgen dependent LAPC4 xenograft, which expresses wild type androgen receptor and produces PSA. The LAPC4 xenograft was grown subcutaneously in a series of immunodeficient mice to a size of 1 cm³. Biopsies were then taken and serum PSA values determined (serum for pretreatment PSA was taken 1 week after the biopsy to avoid artifactual increases due to trauma to the tumor). The mice were then treated with the antisense PMO at 200 μg intraperitonealy (i.p.) daily for 3 days. Serum PSA was then determined on day 4, followed by a second biopsy. In each mouse there was a fall in the LAPC-4 derived PSA of approximately 30-45% (521 to 277, 241 to 138, and 44 to 30 in mice 1-3, respectively) (FIG. 2). In contrast to the human androgen receptor antisense results, PSA levels were stable (in one mouse) or increased (in two mice) with the scrambled oligomer. It should be noted that the serum half-life for PSA in humans is 6 days, but may be more rapid in mice. In any case, these decreases indicate a substantial effect that is specific for the human androgen receptor PMO.

In addition, immunohistochemistry demonstrated a decrease in androgen receptor protein expression in the antisense treated mice (data not shown), which was confirmed by androgen receptor immunoblotting of tumor biopsies taken pre- and post-treatment (FIG. 3). The immunoblots showed marked differences in levels of androgen receptor protein expression that correlated with the PSA levels. LAPC-4 tumor bearing mice were also treated with the scrambled control PMO. Treatments were identical to the antisense treatments and serum PSA was determined immediately pretreatment and one day post treatment (day 4). These results indicate that the androgen receptor antisense PMOs are effective and specific in vivo at down-regulating androgen receptor protein.

### Example 3

### Effect of Androgen Receptor Antisense PMO on Androgen Independent Xenografts In Vivo

An androgen independent prostate cancer xenograft was used to determine whether the androgen receptor antisense PMO could similarly down-regulate androgen receptor expression at this stage of disease. An androgen independent LAPC-4 xenograft was generated by castrating mice bearing androgen dependent LAPC-4 xenografts. A recurrent tumor was then excised, disrupted and re-implanted in the flanks of Ncr nude mice, which had already been surgically castrated. Consistent with previous reports, these tumors grew readily in the castrated mice. When they reached a size of at least 1 cm³, incisional biopsies were carried out to determine androgen receptor levels prior to treatment. Mice were then rested for 5-7 days before serum was drawn to determine baseline PSA levels. Treatment with the androgen receptor antisense PMO was then initiated, and the androgen receptor and serum PSA levels were determined at the completion of therapy.

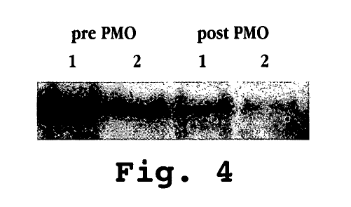

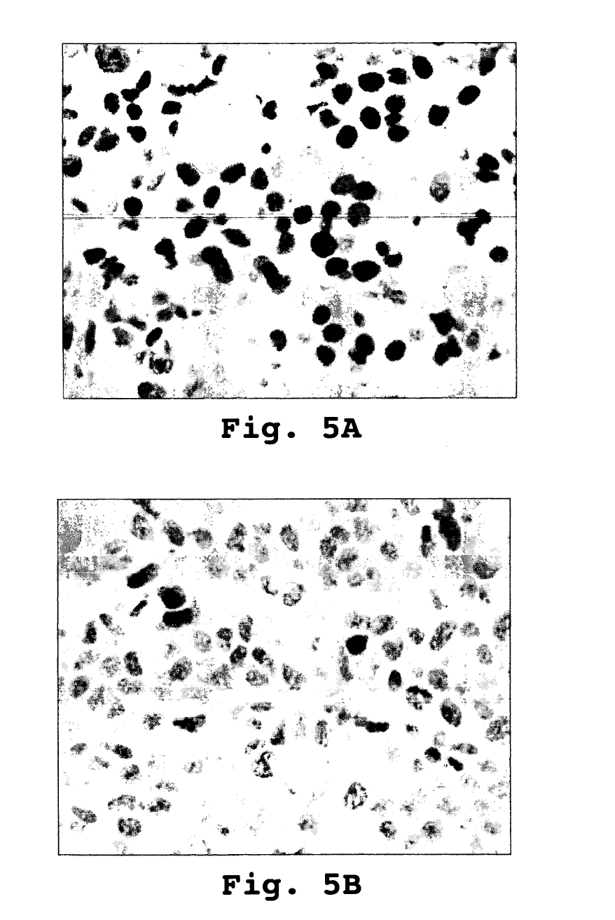

As observed with the androgen dependent LAPC-4 xenografts, the levels of androgen receptor expression in the pretreatment biopsies were variable (FIG. 4). Similar to the results with the androgen dependent tumors, androgen receptor levels were reduced after an extended 14-day course of PMO administration (FIG. 4). Immunohistochemistry was also carried out to determine whether there was relatively uniform decrease in androgen receptor expression versus a decrease in a subpopulation of tumor cells. These results demonstrated that the androgen receptor antisense PMO treatments in these subcutaneous xenografts resulted in a relatively uniform decrease in AR expression, with no evidence for resistant cells still expressing high androgen receptor levels (FIGS. 5A-5B).

### Example 4

### Effect of Androgen Receptor PMO on Androgen Receptor Levels in Normal Mouse Prostate

Two antisense PMO sequences targeted to the mouse and human androgen receptor translational start site and a scrambled mismatch control PMO were synthesized and purified. Purity was >95% as determined by reverse phase HPLC and MALDI TOF mass spectroscopy. The base sequences for the scrambled control are shown and the mispair bases are in bold italics. There is one base mispair between the mouse and human androgen receptor (AR) sequences at nucleotide 7:

| | |
|---|---|
| Mouse AR PMO | |
| 5′-CTGCACCTCCATCCTTGAGC-3′ | (SEQ ID NO:1) |
| Human AR PMO | |
| 5′-CTGCACTTCCATCCTTGAGC-3′ | (SEQ ID NO:2) |
| Scrambled PMO | |
| 5′- CTC GAT CTC ACT CTC GCG AC-3′ | (SEQ ID NO:25) |

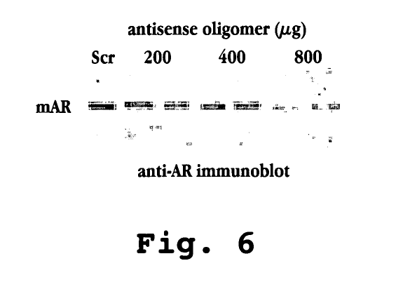

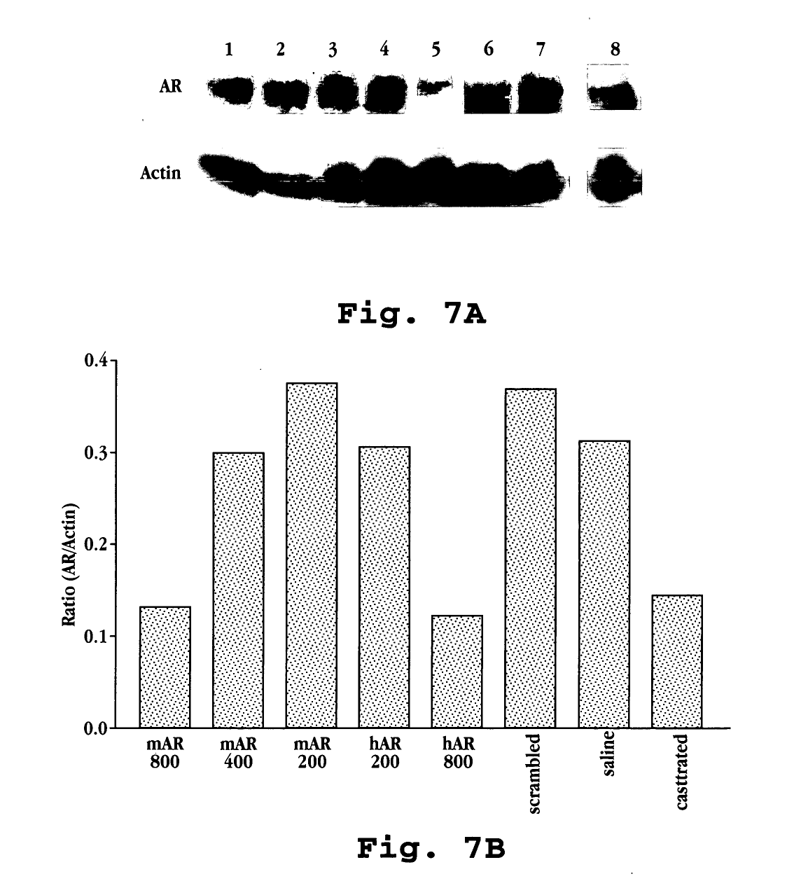

These PMOs were then tested in normal male mice for their ability to downregulate androgen receptor levels in the mouse prostate in two separate experiments. Pharmacokinetics in mice have indicated a half-life of approximately 18 hours, so male mice (129×B6) were treated with single daily intraperitoneal injections for 4 days and prostates were harvested on day 5. As shown by immunoblotting in FIG. 6, the oligomers induced a dose dependent decrease in the expression of androgen receptor protein in ventral prostate. A dose dependent decrease was also observed in seminal vesicle weight, which is a useful indicator of anti-androgen activity (control seminal vesicles were 180 mg, the 200 and 400 μg treated (two mice in each group) averaged 150 mg, and the 800 μg treated mice averaged 110 mg). In a second experiment with age and strain matched ICR mice, both the mouse and human androgen receptor PMO were given to assess their effect on the mouse androgen receptor. The effect of the oligomers was compared to actin as a control. As shown by immunoblotting in FIG. 7A, the oligomers induced a dose dependent decrease in the expression of androgen receptor protein in ventral prostate using either mAR or hAR PMO compared to the level of androgen receptor expression in the prostate of saline or scrambled PMO treated mice. Also shown is the expression of androgen receptor protein in ventral prostate using actin compared to the level of androgen receptor expression in the prostate of saline or scrambled PMO treated mice. As seen in Table 3, a dose dependent decrease was also observed in seminal vesicle weight, which is a useful indicator of anti-androgen activity (control seminal vesicles were 180 mg, the 200 and 400 μg treated (two mice in each group) averaged 150 mg, and the 800 μg treated mice averaged 110 mg). Specifically, the human androgen receptor PMO was also able to decrease the androgen receptor levels in the mouse ventral prostate at the higher 800 μg dose as compared to much higher androgen receptor expression in the saline, castrate and scrambled oligomers. This is an unexpected yet important result as future GMP and GLP toxicity studies in mice can be done using the human androgen receptor PMO instead of using two different sequences.

TABLE 3
Effect of AR Antisense PMO on Seminal Vesicle Weight in Mice

| AR PMO μg/mice | Average Weight (mg) |
|---|---|
| 0 | 180 |
| 200 | 150 |
| 400 | 150 |
| 800 | 110 |

### Example 5

### Bioavailability of Androgen Receptor PMO In Vivo

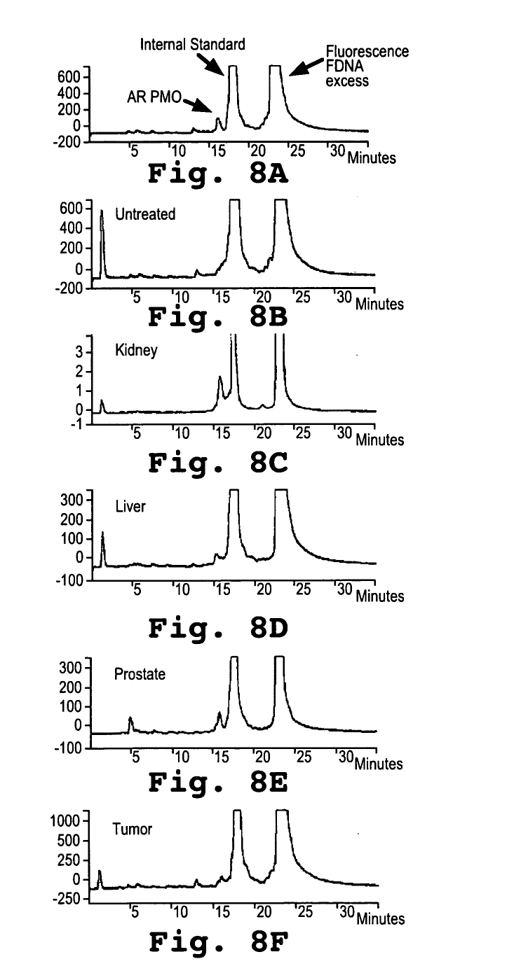

Although the androgen receptor antisense PMO was able to inhibit androgen expression in subcutaneous xenografts, the uptake of the oligomer in this site may be particularly high relative to the prostate or other potential sites for metastatic tumors. To address tissue biodistribution, a series of mice bearing LAPC-4 tumors were treated intraperitoneally with a single dose (400 μg) of human androgen receptor antisense PMO. Mice were then sacrificed 24 hours after PMO administration and tumor, liver, kidney, and prostate tissues were rapidly dissected and snap frozen. Tissue lysates were processed and run on HPLC as described above in the Methods section. The elution order of each chromatogram (FIG. 8) is androgen receptor antisense PMO, the internal standard, and the fluoresceinated DNA probe which is the last to elute. Representative chromatograms to show separation of peaks from the tissue lysates of untreated or androgen receptor PMO treated animals are presented in FIG. 8. The peak corresponding to androgen receptor antisense PMO was observed up to 24 h after administration with a single dose of 400 μg PMO in tumor and prostate samples (FIG. 8 and Table 4). Liver and kidney also showed significant PMO accumulation as illustrated in Table 4 below.

TABLE 4
Androgen Receptor Antisense PMO Concentration in the LAPC4
Androgen-Independent Xenograft Tumors and Organs

| | | PMO | | PMO |
|---|---|---|---|---|
| | Dose | recovered | Total Organ | recovered |
| Tissue | (μg) | μg/g tissue | Weight (g) | μg/organ |
| Tumor | 400 | 0.39 | 0.22 | 0.09 |
| Ventral | 400 | 3.9 | 0.015 | 0.07 |
| prostate | | | | |
| Liver | 400 | 1.93 | 2 | 3.86 |
| Kidney | 400 | 14.93 | 0.28 | 4.18 |

The bioavailability of the AR antisense PMO was further shown in vivo by HPLC analysis. A series of mice bearing LAPC-4 tumors were treated intraperitoneally with a single dose (400 μg or 800 μg) of human androgen receptor antisense PMO. Mice were then sacrificed 24 hours after PMO administration and tumor, kidney, spleen, seminal vesicle, and prostate tissues were rapidly dissected and snap frozen. Tissue lysates were processed and run on HPLC as described above in the Methods section. Androgen receptor antisense PMO was observed up to 24 h after administration with a single dose of 400 μg PMO in tumor, spleen, and prostate samples. Liver, kidney, and seminal vesicle tissues also showed significant PMO accumulation with a single dose of 800 μg PMO as seen in Table 5 below.

TABLE 5
Androgen Receptor Antisense PMO Concentration

| | | PMO recovered |
|---|---|---|
| Tissue | Dose (μg) | μg/g tissue |
| Tumor | 400 | <0.8 |
| Spleen | 400 | <0.8 |
| DL prostate | 400 | 3.9 |
| Ventral prostate | 400 | 4.63 |
| Tumor | 800 | <0.8 |
| Seminal vesicle | 800 | 1.1 |
| Spleen | 800 | 1.2 |
| Prostate | 800 | 2.6 |
| Kidney | 800 | 14.6 |

### Example 6

### Effect of Androgen Receptor Antisense PMO on PSA Levels from Orthotopic Prostate Tumors

An orthotopic prostate tumor model system was used to determine whether the androgen receptor antisense PMO could reduce serum PSA levels in mice bearing LNCaP orthotopic prostate tumors. This prostate tumor model system is established by orthotopic administration of approximately 2×10⁶ LNCaP cells to the mouse prostate. After 20 to 30 days, PSA levels typically increase; an indication of a successful implantation of the tumor cells in the prostate.

Mice whose serum PSA had risen were treated with 400 μg of the androgen receptor antisense PMO (SEQ ID NO:2) daily for five days by intraperitoneal administration. Two mice were treated with PMO and two were untreated controls. Blood was collected in both controls and treated animals before and after the five day treatment period to measure PSA. The following table (Table 6) shows the PSA levels in the prostate orthotopic tumor animals.

TABLE 6
PSA Levels in Prostate Orthotopic Tumor Animals

| | PSA Levels (ng/ml) | PSA Levels (ng/ml) |
|---|---|---|
| Treatment Group | Before Treatment | After Treatment |
| Control Animals | 16.7 | 25.5 |
| (not treated) | 97.9 | 119 |
| Animals Treated with | 21.1 | 16.2 |
| PMO (SEQ ID NO: 2) | 70.4 | 48.8 |

As the above table demonstrates, the androgen receptor PMO decreased the PSA levels in treated mice compared to untreated mice that showed increased PSA levels during the treatment period.

TABLE 7
Sequences

| SEQ | | |
|---|---|---|
| ID | | |
| NO: | Description | Sequence |
| 1 | Antisense oligomer | CTGCACCTCCATCCTTGAGC |
| 2 | Antisense oligomer | CTGCACTTCCATCCTTGAGC |
| 3 | Antisense oligomer | GCACTTCCATCCTTGAGC |
| 4 | Antisense oligomer | GCACTTCCATCCTTGAG |
| 5 | Antisense oligomer | CTGCACTTCCATCCTTGAGCTTC |
| 6 | Antisense oligomer | GTCTGTAGCTTCCACCGAATT |
| 7 | Antisense oligomer | GGCTGAATCTTCCACCTACTT |
| 8 | Antisense oligomer | CCTTCCCAGCCCTAACTGAC |
| 9 | Antisense oligomer | CTTACCGCATGTCCCCGTAAG |
| 10 | Antisense oligomer | CTCCAAACTGGAAAGACAC |
| 11 | Antisense oligomer | GACCCTTTACCTTCAGCGGC |
| 12 | Antisense oligomer | GGTACTTCTGTTTCCCTGGG |
| 13 | Antisense oligomer | GTATCTTACCTCCCAGAGTC |
| 14 | Antisense oligomer | CAGCTTCCGGGCTATTGGG |
| 15 | Antisense oligomer | CCTTTTCCTTACCAGGCAAGGCC |
| 16 | Antisense oligomer | GGAAGCCTGGAGAAGAAGAGG |
| 17 | Antisense oligomer | GCACTTACTCATTGAAAACC |
| 18 | Antisense oligomer | GCATGCGGTACCTGGGAAGG |
| 19 | Antisense oligomer | GGCACTTACTAATGCTGAAG |
| 20 | Antisense oligomer | CCACTGGAACTGATGTGGG |
| 21 | Antisense oligomer | CGTTTGCTTACAGGCTGCAC |
| 22 | Antisense oligomer | CTCGCAATCTGTAGGGAAG |
| 23 | internal standard | GAGGGGCATCGTCGC |
| 24 | internal standard | FAM-GCG ACG ATG CCC CTC AAC GT |
| 25 | scrambled oligomer | CTCGATCTCACTCTCGCGAC |

1. A method of treating prostate cancer in a subject having an androgen-independent prostate cancer, as evidenced by a lack of response in PSA level to androgen-suppression therapy, said method comprising
(a) administering to the subject, an oligonucleotide analog compound characterized by:
(i) 12-40 morpholino subunits,
(ii) a substantially uncharged, phosphorus-containing backbone linking said subunits,
(iii) active uptake by human prostate cancer cells,
(iv) a base sequence that is complementary to a target region containing at least 12 contiguous bases in a preprocessed or processed human androgen receptor transcript, and which includes at least 6 contiguous bases of the sequence selected from the group consisting of: SEQ ID NOS:2, 7, 8, and 9-22, and
(v) capable of hybridizing with a preprocessed human androgen-receptor transcript to form a heteroduplex structure having a Tm of dissociation of at least 45° C.,
(c) following said administering, monitoring the subject's serum PSA level, and
(d) continuing said administering, on a periodic basis, at least until a substantial drop in the subject's serum PSA level is observed.

2. The method of claim 1, wherein the compound administered is composed of morpholino subunits linked by uncharged, phosphorus-containing intersubunit linkages, joining a morpholino nitrogen of one subunit to a 5′ exocyclic carbon of an adjacent subunit.

3. The method of claim 2, wherein the intersubunit linkages in the compound administered are phosphorodiamidate linkages.

4. The method of claim 3, wherein the morpholino subunits in the compound administered are joined by phosphorodiamidate linkages, in accordance with the structure: 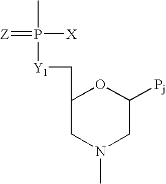
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

5. The method of claim 4, wherein X=NR₂, where each R is independently hydrogen or methyl in the compound administered.

6. The method of claim 1, wherein the compound administered is effective to target the start site of the processed human androgen start site and has a base sequence that is complementary to a target region containing at least 12 contiguous bases in a processed human androgen receptor transcript, and which includes at least 6 contiguous bases of the sequence selected from the group consisting of: SEQ ID NOS:2, 7, 8.

7. The method of claim 6, wherein the compound administered includes a base sequence selected from the group consisting of: SEQ ID NOS:2, 3, 4, 5, 7, and 8.

8. The method of claim 1, wherein the compound administered is effective to target a splice site of preprocessed human androgen start site and has a base sequence that is complementary to a target region containing at least 12 contiguous bases in a processed human androgen receptor transcript, and which includes at least 6 contiguous bases of the sequence selected from the group consisting of: SEQ ID NOS:9-22.

9. The method of claim 8, wherein the compound administered includes a base sequence selected from the group consisting of: SEQ ID NOS:9-22.

10. The method of claim 1, which further includes administering a chemotherapeutic agent to the subject.

11. The method of claim 1, which further includes, at a selected time after said administering, obtaining a sample of a body fluid from the subject; and
assaying the sample for the presence of a nuclease-resistant heteroduplex comprising the oligonucleotide analog compound complexed with a complementary portion of a preprocessed human androgen receptor transcript.

12. An oligonucleotide analog compound for use in treating prostate cancer in a subject, characterized by:
(i) 12-40 morpholino subunits,
(ii) a substantially uncharged, phosphorus-containing backbone linking said subunits,
(iii) active uptake by human prostate cancer cells,
(iv) a base sequence that is complementary to a target region containing at least 12 contiguous bases in a preprocessed or processed human androgen receptor transcript, and which includes at least 6 contiguous bases of the sequence selected from the group consisting of: SEQ ID NOS:2, 7, 8, and 9-22, and
(v) capable of hybridizing with a preprocessed human androgen-receptor transcript to form a heteroduplex structure having a Tm of dissociation of at least 45° C.

13. The compound of claim 12, which is composed of morpholino subunits linked by uncharged, phosphorus-containing intersubunit linkages, joining a morpholino nitrogen of one subunit to a 5′ exocyclic carbon of an adjacent subunit.

14. The compound of claim 13, wherein said intersubunit linkages are phosphorodiamidate linkages.

15. The compound of claim 14, wherein said morpholino subunits are joined by phosphorodiamidate linkages, in accordance with the structure: 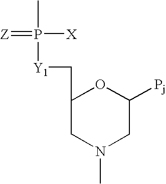
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

16. The compound of claim 15, wherein X═NR₂, where each R is independently hydrogen or methyl.

17. The compound of claim 12, which is effective to target the start site of the processed human androgen start site and which has a base sequence that is complementary to a target region containing at least 12 contiguous bases in a processed human androgen receptor transcript, and which includes at least 6 contiguous bases of the sequence selected from the group consisting of: SEQ ID NOS:2, 7, 8.

18. The compound of claim 17, which includes a base sequence selected from the group consisting of: SEQ ID NOS:2, 3, 4, 5, 7, and 8.

19. The compound of claim 12, which is effective to target a splice site of preprocessed human androgen start site and which has a base sequence that is complementary to a target region containing at least 12 contiguous bases in a processed human androgen receptor transcript, and which includes at least 6 contiguous bases of the sequence selected from the group consisting of: SEQ ID NOS:9-22.

20. The compound of claim 19, which includes a base sequence selected from the group consisting of: SEQ ID NOS:9-22.

21 The compound of claim 12, in a composition which also includes a chemotherapeutic agent.

22. A method of confirming the presence of an effective interaction between a human androgen-receptor pre-processed transcript and an uncharged morpholino oligonucleotide analog compound, comprising
(a) administering said compound to the subject, where said compound is characterized by: (i) 12-40 morpholino subunits, (ii) a substantially uncharged, phosphorus-containing backbone linking said subunits, (iii) active uptake by human prostate cancer cells, (iv) a base sequence that is complementary to a target region containing at least 12 contiguous bases in a preprocessed human androgen receptor transcript, and (v) capable of hybridizing with a preprocessed human androgen-receptor transcript to form a heteroduplex structure having a Tm of dissociation of at least 45° C.,
(b) at a selected time after said administering, obtaining a sample of a body fluid from the subject; and
(c) assaying the sample for the presence of a nuclease-resistant heteroduplex comprising the oligonucleotide analog compound complexed with a complementary-sequence portion of a preprocessed human androgen-receptor transcript.
